(19) United States
(12) Patent Application Publication
(10) Pub. No.: US 2010/0191203 A1
(43) Pub. Date: Jul. 29, 2010

(54) System and equipment for persons with stoma

(75) Inventor: Hans H. HARALDSTED, Farum (DK)

Correspondence Address: HANS H. HARALDSTED, POSTBOX 107, FARUM, DK3520, DK

(73) Assignee: King Yuan Electronics Co., Ltd., Hsinchu (TW)

(21) Appl. No.: 12/460,094

(22) Filed: Jul. 10, 2009

(30) Foreign Application Priority Data
Jan. 27, 2009 (DK) PA 2009 00116

(51) Int. Cl. A61F 5/448; A61F 5/445

(52) U.S. Cl. 604/343; 604/338

(57) ABSTRACT

An ostomy appliance for a single ostomy pouch, alternative a couple able pouch, namely an ostomy appliance as a pressure ring; said a flat cylindrical pressure ring, with an outer circularly diameter, and an inner circularly diameter; said which inner circularly diameter in its size is made or chosen so it fit, and touch to the surface of the actually stoma, and where the pressure ring is hold and coupled floating to the system.

### THE AREA OF THE USE OF THE INVENTION

The invention concern a system and equipment for optimising, and with this, improvement the conditions by equipment there has been used of persons with stoma, or persons who have got the intestine or other liquid giving organs operated out through the abdominal cavity or by analogous conditions.

### KNOWN TECHNIQUE

It is known from U.S. Pat. No. 5,429,625, made of Mr. Steen Holmberg of Hollister/Dansac,—to use a solution for ostomy, with a conical formed pressure ring

The pressure ring is here mounted on and between the coupling ring of the bag, and on the coupling ring of skin-plate. The pressure ring is mounted between the two couplings systems, and is fixed firmed to this system.

The disadvantage by these systems is for the first, that the conical pressure ring will allow and caused that the rim of skin protection plate around the stomi hole will swipe out and open for the skin tight to the rod of the stoma, because it will touch with a very small rim, edge or tip of the pressure ring.

There will also be a little disadvantage more, because the US system is build high, next because it or the pressure ring here is hold firmed for the whole system, it will not be possible to make a form of pumping system, which continuous will be able to pump fresh skin protection means against the rod of the stoma.

The next disadvantage is also that this pressure ring do not in the hole is so small that the rim of the hole touch the stoma, and by this also has to be better to press a skin protection means against the rod of the stoma.

And next the US system is not floating mounted and self centring, and therefore can this solution not be able to be used as pumping system, and a system, which ring is touching the stoma for high tightening.

It is also known, that one has got arrangements of bags, where one outer have got a cuff with a specific size of hole. And where the bag firmed has been narrowed in such a way that the cuff has to be able to be resting on the sticking round and presses around the stoma.

The disadvantage by this system has been that one, because of special system, and because of the outer cuff with only one size of hole, has been obliged to go on compromise so that the hole of the cuff itself was much bigger than the stoma itself. And further the embodiment caused that the cuff itself has been able to be displaced or moved, and in unlucky cases by this has to be able to squeeze on the stoma itself.

The mentioned arrangement of cuff has separated been hold tight to the stoma by an arrangement of a belt, which was coupled to the cuff itself.

### THE TECHNICAL PROBLEM THERE HAS TO BE SOLVED

With the invention one want to make a system and equipment, which optimise the secure against that the equipment leak. But in the same time also cause that the mounted equipment keep much longer, and that the user, not at a wrong moment as earlier got unpleasant disturbs with destroyed skin, so that a mounting of that reason will be very much difficult to do or make, for a reasonable satisfaction and securing.

### THE NEW TECHNIQUE

The new is that the system consists of a pressing ring, which is flowing mounted and in direct intern connection with the stoma itself, and that the pressing ring in its arrangement of mounting and the way of this is self-centring around the stoma. And that the pressure ring fit separate to and around in its size of the hole for the actually stoma. And that the pressure ring is made of bending and partly springy material, but still firm material, which in the same time do not are soluble in those mediums and substances they are surrounded of. And where the firm pressure ring as an example is made of plastic, steel, metal or other like materials with like properties.

One obtain by this that for the first that the skin protection plate itself by the firm and intern placed pressure ring then will be able to hold quit tight and compact against the belly skin. And here especially around the critically area of skin itself, where the stoma is going through. As the firm pressure ring here just in this system and embodiment has a size of hole there fit special to the actual stoma, and to that it is mounted intern almost to or often for touching with the surface of the stoma itself.

There has also been obtained that the skin protection plate or it has to be a skin protection ring that it will be able to hold or stand longer, as it only will be the edge or the thickness of the plate there will be influenced of the break down material which stoma gives.

And further one obtain by the system, with the firm and intern placed ring, and with the actual size of the hole that one will be able to minimise the problem the has to be obtained, when the person twist and bow him or her self. Which often earlier caused that the skin plate or the ring slip its contact to the skin of the belly, and then allow critical material from the stoma to creep under and gives damages on the skin. But also in the same time loosen the whole arrangement of bag from the skin, which caused that the whole arrangement should be changed.

The new is also that the system for a “floating mounting”, and one with this integration of the pressure plate consisting of a coupling plate, with a coupling ring for mounting of a loos bag, also with a coupling ring (male or female).

The coupling ring on the coupling plate is coupled together via a flexible and movable bellows.

The bellows itself in the system is by this going to be “ bearing house” for the pressure plate.

The coupling ring on the bellow is stop for the pressure plate.

The coupling plate is in axial direction opposite stop.

The diameter of the arrangement by the coupling ring is smaller, than the outer diameter of the pressure plate and made therefore a stop. And further in the same time, is the bellow of the system establish a floating mounting, where this floating mounting constant therefore made or done a self-centring specially around the stoma itself.

One obtain by this that the mounted pressure ring ideally will be able to be mounted intern in the system of the bag, and in contact with the stoma itself, and in contact with the means of skin protection, as skin plates- or skin protection rings.

One obtain also opposite earlier, where one only have extern pressure rings that the skin protection ring can be able to come tight against the rod of the stoma. And in the same time, qua its mounting, one obtain also in an arrangement of bellow that the mounted pressure ring constant will be self centring around the stoma, and not in unlucky cases, where the extern mounted pressure ring cause that this glide down over and on the stoma.

There has also been obtained that the pressure ring itself will be able capsize like a cardan joint, so that even a person turn his or her body, then will the pressure plate constant hold compact at the surface in such a way that the system do not will leak, even if one of the sides on the pressure ring slip its contact with the skin protection plate, or it has even also be at simple cases, where one only has stick or glued the bag direct on the skin, the will the arrangement of bag also be able to hold or keep better.

The new is also, that the pressure ring is made of materials, which have such springy conditions, and size in the outer diameter and in thickness of the goods,—that this or the pressure ring by mounting very easy and unproblematic can be turned, as an example via ideally a bending of the pressure ring itself into a pipe-shape bellow arrangement, and after this pop back in its original plane level for a self fixing, where the road or the passage of the admission to this is lesser in diameter than the outer diameter of the pressure plate. And that the thickness of the pressure ring has a comfortable thickness, so that the inner edge, which is proper rounded, and which touch the stoma do not will be able to cut on the surface of stoma. But under this also in such that the outer diameter of the pressure ring is rounded in such a way and manner, that the pressure ring do not will be able to cut in the bellow or the bag. But in the same time also with such a thickness that the plane plan, which the ring formed that it will be rather firmed and stable for its here necessary plane pressure function on the skin protection plate and so.

The new is also that the system, with the floating mounted pressure ring in alternative embodiment has to be coupled together with an arrangement of partly fluid and formable packing consisting of a module-able skin protection ring. And which system and packing has to be mounted on the side there is turned against the belly. And following be mounted around the stoma, where the following the inner diameter of the firm pressure ring by this will hold the form-able skin protection ring against the critical edge or passage between the stoma and the belly skin for an optimum sealing and skin protection.

One have obtained by this that the coupled system itself together will optimise the effect of a moulded skin protection ring and the firm pressure ring itself, as the system constant will be hold or pressed compact to the surface of the belly skin. In such a way that a leak by leaking of material from the stoma under the skin protection ring and the skin protection plate, which the arrangement of bag is couple to, will be avoided.

The new is also that the pressure ring in its floating mounting in the movable coupling bellow itself by moving by the user of this system, by this, establish a sort of a pumping effect by its turning of the pressure ring forward and backward around the stoma itself. And in such a way that the material or the mass of the skin protection ring continuous against the rod itself of the stoma successive pump or squeeze against here.

One obtain by this that the partly flowing skin protection mass continuous will be pressed against and around the rod itself on the stoma, by which the possibility also continuous dissolve skin protection mass will be renewed.

The new is also that the single stoma bag, alternative the single skin plate in an alternative embodiment has to be made with a bellow- or a pocket arrangement for mounting or integrating of a firm pressure ring.

The has to be obtained that the earlier normally known system, with bags and skin protection plates, also will be able to use this facilitate there are by coupling of a firm pressure ring to this systems. Because one obtain that the pressure ring both will be able to hold the system compact and secured against the skin area on where it is mounted. But perhaps also that one in the same time obtained a sort of a pumping effect, so that one automatically get the mass of the skin plate pressed continuous against the tight edge and the area at the stoma.

The new is also that the pressure ring has to be used without a floating mounting, by, that it alternative has to be mounted direct on a skin protection ring or a plate for stabilising and compact and smooth hold of a skin protection-ring or plate.

One obtain by this that the arrangement of skin protection as a skin plate or a skin protection ring, or perhaps only a skin protection pasta, will be hold compact against the critical skin area, where it has to be laid.

One obtain by this a better and more durable effect or a more durable arrangement in the actually area.

The new is also that the pressure ring is equipped with holes, which either are empty or, which are filled up with skin protection mass. And where these holes either can be through-going or be made as cupping, or alternative as net-patterns, and which via its roughness and qua this arrangement then has to establish a firmed counter hold, where the pressure ring has to be placed against.

One obtain by this that one combine both the properties of the firm ring, and in the same time with that the skin protection mass has to be dosed or hold against the actually. Why the arrangement will be lesser critical as regards to couple two different systems together, with different properties.

The new is also that the loose firm pressure ring, which in its size of hole for the stoma, has to be fitted to the actually user, in an rational embodiment into suitable springs or steps, as an example on 3 mm or 5 mm.

And where a practical belonging stoma diameter or organ diameter can goes from 15 mm to 45 mm, namely so that there by step on 5 mm only shall be fabricated 6 different rings, or a little more by step on 3 mm.

One obtained by this that the firm ring, which not direct will be possible to prepared or worked via as an example a normally scissors or like, and further where there shall be a probably exact size of hole. Then in a practical embodiment will be able to be made in reasonable few issues and with suitable step or changes in the size of the hole. Which both done the system fair economic in its embodiment, and in the same time practical and easy and unproblematic to use.

The new is also that the pressure ring has all the sizes of hole integrated by that the different sizes of holes are established by breakable, but connected and coupled rings, which can be break off and from each other.

One obtain by this that the user only shall buy a larger pressure ring, and then after this, can he or her break into the correct size of hole, which the actually pressure shall have.

The pressure ring will also be cheaper to fabricate in one model in stead of more models depending on the actually size of the hole.

### TEST EXPERIMENT OF THE INVENTION

There has now for longer times on a year been made a very successful and a positive test with the invention on a user, who is ileo-stomist.

There shall in this relation be mentioned that an ileo-stomist is one of the most critical user of stoma equipment, because of the aggressive material as thin faces with more, which come direct from the small intestine, because the large intestine is removed.

The result has here in connection by the test experiment of the invention been,—that the skin around the stoma under the test period has been very well-preserved and soft and smooth. And furthermore without damages as wounds, and flushing and more, which other earlier stoma equipment generally always more and less has caused.

Another positive factor by the invention is that the equipment now keeps secured for the user in much more days before it shall be changed.

The invention cause in the same time that it has been much lesser complicated to mount the equipment correct for the user, as the invention cause that there has been much more tolerance on adjustment of the equipment for the actual user.

This is special valid for adjustment around the stoma. As the tolerance before was that one should fit or adjust the size of the hole to be extreme tight laying against the rod of the stoma, to obtain less possible for risk for leak from here.

By tests there has been used equipment from the American firm Hollister, where one have used theirs skin plates with a coupling arrangement, which is mounted on the top of a closed tight pipe shaped bellow.

The bellow arrangement in the Hollister product has been used according to the staging of the actual invention to quit another purpose than it is think original. As it or the bellow arrangement in combination with a firm pressure ring, which is a part of the invention, use the inner pipe shape cavity of the bellow arrangement as flexible and guiding and self-centring holding arrangement. Why this new combination after the very best thought will be quite new and therefore will be able to get a patent. As it is not nearby for an expert into the actually technique only to think this combination and solution, as a normally technical routine in the métier or business.

In the connection with the coupling plate and the pressure ring of the invention, which has to be mounted in the front laying pip shape short arrangement of bellow. Then will a supplying soft and gluing and formable skin protection ring optimise the result of the user of the invention, as the formable skin protection ring has to be laid or mounted on the side there turn against the skin and edge of the stoma. And has to be glued in the first time to the flat of the coupling plate against the skin. As it still has to be laid in such a way that it will goes over the edge of the clipped hole in the coupling plate and the pressure ring, so that this very well lay some millimetre freely.

The hole in the coupling plate and in the pressure ring of the invention will ideally be able to be round as a total circle. Why one only have to fit the edge of the hole via its soft skin protection ring, even the hole and the stoma is shaped oval.

By tests it has just shown to be optimum, when the hole in the pressure ring and in the coupling plate has to be cut larger as 2- to 5 mm the whole way round. This cause with the belonging floating mounting of the pressure ring that the arrangement running or continuous will press the material from the skin protection plate against the middle and the edge of the stoma rod for an optimum tightening and protection.

The “floating” pressure ring will establish a sort of a piston arrangement, by which one obtain an indirect pumping effect on the soft mass, as the skin protection-ring consist off. As the soft mass of the skin protection ring has to be pressed out at the stoma, because here there will be space. While the outer edge on the coupling plate has to stick or fix to the skin, and this way get resistance for an exhausting of mass this way

Opposite it is tried to cut the hole in the coupling plate so it fit tight against the lowest rim of the stoma. Which the result that the material from the soft skin protection ring in stead of to be pressed against the stoma, then has to be pressed out against the outer edge of the coupling plate, by which that this inexpedient has to be loosed.

Another disadvantage was also, by cutting the hole so that it fit tight against the stoma,—that the edge of the hole on the upper firm and thin edge of the coupling plate,—and where this coupling plate do not consist of skin protection material. That this could be able to cut into the stoma and bother this very much with open wounds and like.

They materials there were used by the test, which test still continuous because the optimal conditions they give, are all from the manufacture Hollister Inc. from US.

Formally it shall otherwise be remarked in this patent document that the solution and the invention is shown “in a closed group and into a strong confidence” for Hollister both in Denmark and in US. Who in this special forum and under these specially conditions have shown very much interest and attention for the invention and its complex of idea and its possibilities of developments.

The invention has under above-mentioned conditions been shown to the firm itself, as a working “proto-type”, and in an earlier made and confidential Danish applied patent document with number (PA 2008 0799), applied the date 10 Jun. 2008.

Back to the question about the materials there have been used, or can be used for establishing of the invention as described according to enclosed patent document. Then can one of course also use other material and like products, which more and less could be modified after the actually conditions and circumstances.

### LIST OF FIGURES

The invention shall after this be explained closer, and under reference to the drawing, where

FIG. 1 shown, seen from the front, one at the user mounted stoma equipment, where the stoma equipment consist of a supporting skin plate 3 with a coupling 2 for mounting of a bag 4, and where the supporting skin plate 3 via a coupling arrangement 2 also has equipment for a belt 5.

On the drawing is shown, how a firm pressure ring 1, with a size of the hole which fit to the actually user has to be mounted under the couplings ring 2, but in front off the supporting skin plate 3.

The coupling 2 in this embodiment is not placed tight on, or compact with the skin plate 3. But the coupling 2 is placed via a short pipe- and bellow arrangement 11 there lift the coupling 2 a little from the plate 3, but in the same time does that the coupling 2 partly will be partly yielding or a little movable.

It is the space behind the coupling ring 2 there has to be uses as a fixing- and holding arrangement for the pressure ring 1.

The shown bag 4, which is drawn with a dot-and-dash line is for that matter possible to be drained.

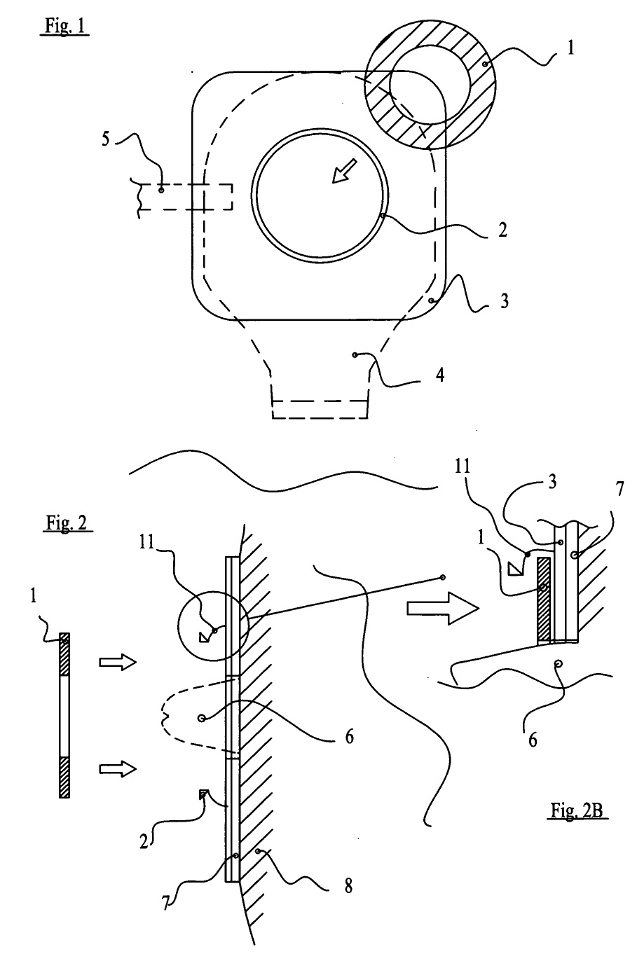

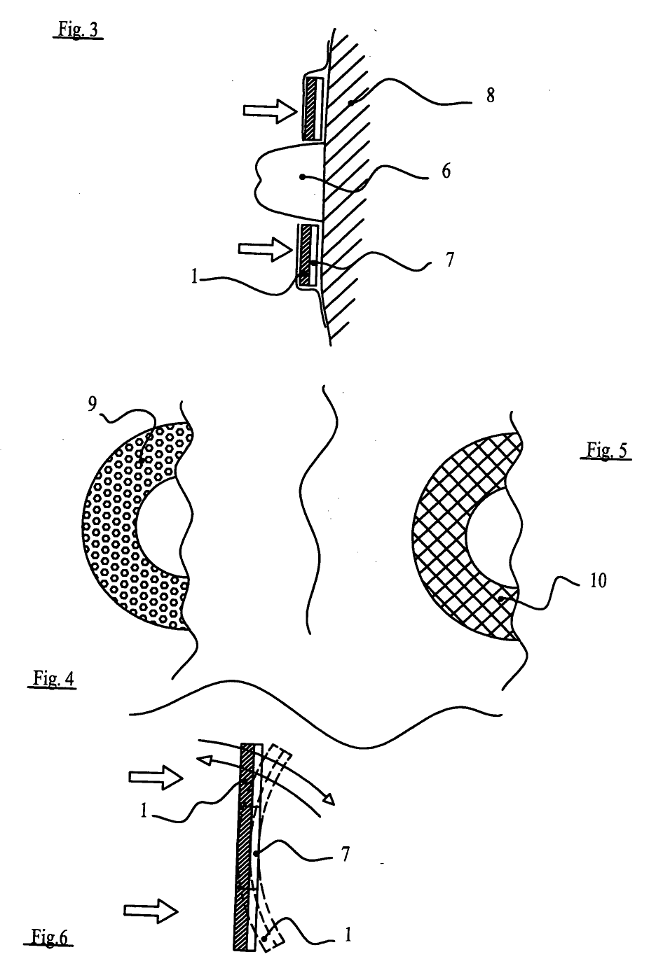

FIG. 2 shown, seen from the side and into cut the same arrangement as shown in FIG. 1, where there here very clearly can be seen the stoma 6 and the pipe- and bellow arrangement 11, where also the firm pressure ring 1 has to be mounted. Said in machine language “ loose” and “ flowing”, but still via this arrangement 1 and 11 and combination 1 and 11 has to be hold and centred around the stoma 6.

The arrangement 1 and 11 will into a lesser grade after the proportions be able to turn a little, or said more specified to be “guided” yielding, in such a way to optimal proportions can be reached for the arrangement 1 and 11.

FIG. 2B shown, and seen into a cut, and seen from the side in detail, how that the firm pressure ring 1 is mounted under the bellow 11 between the coupling ring 2, and the skin plate 7. But it shows too how the pressure ring 1 is guided 11 and firmed placed around and tight on the stoma 6.

FIG. 3 shown, seen into a cut and from the side, an alternative embodiment of the arrangement and according to the system, how the firm pressure ring 1 is placed under the gluing flat of the stoma, but above on a skin plate ring 7.

The pressure ring 1 itself will via its firm form cause that it 1 has to be hold tight to the skin 8 and against the rod of the stoma 6.

The advantage by this arrangement is that the skin plate arrangement 7 gets lesser possibility for leaving the contact and the laying against the skin 8, and the area around the stoma 6, including to avoid a leaking in off substances, which comes from the stoma 6.

FIG. 4 shows, in cut and seen from front, a firm stoma ring 1, which is equipped with a lot off holes 9 or cupping 9. Where the holes 9 will be able to get a skin plate material 7 or a skin paste or like.

FIG. 5 shows, in a cut and seen from the front a stoma ring 1 with a material quality shaped in a net form 10, alternative, where the net form only is a rough surface 10.

The same conditions, which is present under FIG. 4 will also be able to be used here, or is valid here.

FIG. 6 shows, in a cut and seen from the side a firm pressure ring 1, which one has integrated with a skin plate ring 7. Either where this is a permanent solution as a whole 1 and 7, or where the two rings 1 and 7 by use are gathered or integrated in each other 1 and 7, by which one obtain a stability qua the firm ring 1, combined by a ring made of skin protection material 7, which will be ideally for the skin 8.

1. System and equipment (1) for optimising and with this bettering of the physical proportions by equipment there has been used of persons with stoma, or people there have got their intestine (6) or other liquid giving organs (6) operated out through the abdominal cavity (8) or by similar proportions, characteristic in that the system consist of a pressure ring (1), which is floating mounted, and in the same time in direct intern contact with the stoma itself (6), and that the pressure ring (1) in its montage arrangement and way is self centring around the stoma (6), and that the pressure ring (1) fit separate to and around the size of the hole to the actually stoma (6), and that the pressure ring (1) is made of bending-able and partly flexible material, but still firm material, and which is not soluble in the mediums and stuffs the are surrounded in, where the firm pressure ring (1) as an example is made of plastic, steel, metal or other similar materials with mentioned properties.

2. System and equipment (1) for optimising and with this bettering of the physical proportions by equipment there has been used of persons with stoma according to claim 1, characteristic in that the system for a floating mounting, and one by this integration of a pressure ring (1) consist of a coupling plate(3) with a coupling rim (2) for mounting of a loos bag (4), also with a coupling rim (2) (he- or her couplings arrangement (2)), and where the coupling rim (2) on the coupling plate (3) is coupled together via a flexible and movable bellow, where the bellow itself in the system is or by this will be “bearing house” for the pressure ring (1), and where the coupling rim (2) on the bellow is stop for the pressure ring, and where the coupling plate (3) is stop opposite in the axial direction, because the diameter by the arrangement and by the couplings rim (2) is lesser here than the outer diameter for the pressure ring (1), and where in the same time the bellow by the system establish a floating mounting, which is constant self centring around the stoma (6).

3. System and equipment (1) for optimising and with this bettering of the physical proportions by equipment there has been used of persons with stoma according to above mentioned claims, characteristic in that the pressure ring (1) has been made of material, which has such springy properties and size in the outer diameter and in thickness of goods that it by mounting easy and unproblematic can be turned, as an example, ideally via bending (as shown in FIG. 6) into the bellow arrangement and after this pop back to its ordinary plane plan for a self fixing, and that the thickness of the pressure ring (1) has a right or comfortable thickness, so that the inner edge, which is right rounded and which touch the stoma (6) do not will be able to cut in the surface of the stoma (6), under here also in such a way that the outer diameter of the pressure ring (1) is rounded, so that the pressure ring (1) do not can cut into the bellow or the bag (4), but also made with such a thickness that the plane plan itself, which the pressure ring (1) form, will be firmed and stable for it function of pressure.

4. System and equipment (1) for optimising and with this bettering of the physical proportions by equipment there has been used of persons with stoma according to above mentioned claims, characteristic in that the system by the floating mounting of the pressure ring (1) in an alternative embodiment has to be coupled together with an arrangement of a partly fluid and formable packing (7), consist of a formable ski protection ring, which is mounted on that side there is turning against the belly skin (8) and around the stoma (6), and where the inner diameter of the firm pressure ring (1) by mounting will hold the formable skin protection ring (7) against the critical edge or rind between the stoma (6) and the belly skin (8) for an optimal sealing and skin protection.

5. System and equipment (1) for optimising and with this bettering of the physical proportions by equipment there has been used of persons with stoma according to above mentioned claims, characteristic in that pressure ring (1) in its floating mounting in the movable coupling bellow itself by moving of the user (8) in this system (1) by this establish a pumping function by its rocking of the pressure ring (1) backwards and forwards around the stoma itself (6), so that the material (7) or the mass of the sin protection ring (7) continuous has to be pumped or squeezed against the rod of the stoma itself (6).

6. System and equipment (1) for optimising and with this bettering of the physical proportions by equipment there has been used of persons with stoma according to above mentioned claims, characteristic in that the single stoma bag (4), as the single skin plate (3) in an alternative embodiment has to be made with a bellow- or a pocket arrangement, for mounting or integrating of a firm pressure ring (1).

7. System and equipment (1) for optimising and with this bettering of the physical proportions by equipment there has been used of persons with stoma according to above mentioned claims, characteristic in that the pressure ring (1) has to be used without a floating mounting, by that it (1) has to be mounted direct on skin protection ring (7), or a plate (7) for establishing of a stabilising and a plane holding of a skin protection ring (7)—or a plate (7).

8. System and equipment (1) for optimising and with this bettering of the physical proportions by equipment there has been used of persons with stoma according to above mentioned claims, characteristic in that the pressure ring (1) is equipped with holes (9), which either are empty or which are filled up with skin protection mass (7), and that these holes (9) can be through going or be made as cupping, or alternative as netting design, and which via its roughness (9) qua this arrangement (9) then will establish a stable counter-hold for the pressure ring (1) itself, and where it has to be placed against (8).

9. System and equipment (1) for optimising and with this bettering of the physical proportions by equipment there has been used of persons with stoma according to above mentioned claims, characteristic in that the loose, but still the firm pressure ring (1), which in the size of holes for the stoma (6) fit to the actually user into a rationally embodiment, and in suitable spring on as an example 2 to 10 mm, where a practically belonging stoma diameter or organ diameter can go from Ø15 mm to Ø45 mm, namely in such that there only by spring on as an example 5 mm only shall be fabricated 6 different rings, or a little more by spring with lesser size of holes.

10. System and equipment (1) for optimising and with this bettering of the physical proportions by equipment there has been used of persons with stoma according to above mentioned claims, characteristic in that the pressure ring (1) has all sizes of holes integrated by, that different exact sizes of holes are established by crack-able, but connected and jointed rings, which at need can be cracked from each other, and by this fitted for the actually user with the actual size of stoma (6).
